(19) United States
(12) Patent Application Publication
(10) Pub. No.: US 2015/0157653 A1
(43) Pub. Date: Jun. 11, 2015

(54) Prevention of Clostridium Difficile Infection in High Risk Patients

(71) Applicant: Optimer Pharmaceuticals, Inc., Lexington, MA (US)

(72) Inventors: Sherwood Gorbach, San Diego, CA (US); Pamela Sears, San Diego, CA (US); Yoshi Ichikawa, San Diego, CA (US)

(73) Assignee: Optimer Pharmaceuticals, Inc., Lexington, MA (US)

(21) Appl. No.: 14/385,744

(22) PCT Filed: Mar. 15, 2013
(86) PCT No.: PCT/US13/32249

§ 371 Date: Sep. 16, 2014

Related U.S. Application Data
(60) Provisional application No. 61/611,965, filed on Mar. 16, 2012.

(51) Int. Cl. A61K 31/7048; A61K 45/06

(52) U.S. Cl. CPC A61K 31/7048; A61K 45/06

(57) ABSTRACT

The present invention relates to methods of preventing Clostridium difficile infection (COI) in a human subject who is at high risk of developing this infection, comprising administering to the subject an effective amount of the compounds described herein. In some embodiments, the subject is further receiving concomitant antibiotic therapy to prevent and/or treat an infection other than Clostridium. In some embodiments, the subject previously was treated for a Clostridium infection. In some embodiments, the compound of formula I prevents the Clostridium infection.

### FIELD OF THE INVENTION

The present invention relates to methods of preventing Clostridium difficile infection (CDI) in a human subject who is at high risk of developing this infection, comprising administering to the subject an effective amount of the compounds described herein.

### BACKGROUND OF THE INVENTION

Clostridium difficile (C. difficile) is an anaerobic spore-forming bacterium that causes an infection of the bowel. Diarrhea is the most common symptom, but abdominal pain and fever may also occur. C. difficile is a major causative agent of colitis (inflammation of the colon) and diarrhea that may occur following antibiotic intake. This bacterium is primarily acquired in hospitals and long term care facilities.

Clostridium difficile infection (CDI) or C. difficile-associated diarrhea (CDAD) is a disease characterized by severe and painful diarrhea. C. difficile is responsible for approximately 20% of the cases of antibiotic-associated diarrhea (AAD) and the majority of the cases of antibiotic-associated colitis (AAC).

C. difficile is a gram positive anaerobe that causes diarrhea through production of at least two toxins, A and B, in the colon of infected individuals. The predominant risk factor for CDI is broad-spectrum antibiotic use. Recently, the incidence, severity, and recurrence rate of CDI has increased. The emergence of a hypervirulent C. difficile strain first described in 2000, known as NAP1, has partially contributed to the changing epidemiology of this disease.

Diarrhea is a frequent and debilitating complication after hematopoietic stem cell therapy (HSCT). HSCT recipients are known to be at increased risk for a wide variety of infectious complications, including CDI as a result of prolonged hospitalizations, exposure to broad-spectrum antibiotics, altered integrity of the intestinal mucosa and graft versus host disease (GVHD). The incidence of CDI in the HSCT population has been reported as high as 20% in some large series.

These recent studies point out the high risk of CDI in HSCT recipients using current conditioning and prophylactic measures. The wide-spread inclusion of antibacterial agents such as trimethoprim, carbapenems and a fluoroquinolone may contribute to the current high risk of CDI. In addition, the immunocompromised state is an additional risk factor.

In HSCT, prophylaxis against infectious agents during the period of highest risk for infection has proven effective in reducing the incidence of infectious complications in this high risk patient. An ideal agent for CDI prophylaxis in high risk patients would have the properties of tolerability, a narrow spectrum of activity, bactericidal, no cross resistance between other agents used to treat CDI and ability to be taken orally and little systemic absorption. There are currently two dominant therapies for CDI: vancomycin and metronidazole. Vancomycin is not recommended for first-line treatment of CDI mainly because it is the only antibiotic active against some serious life-threatening multi-drug resistant bacteria. Therefore, in an effort to minimize the emergence of vancomycin-resistant Enterococcus (VRE) or vancomycin-resistant S. aureus (VRSA), the use of this drug is discouraged except when absolutely necessary.

Tiacumicins, specifically Tiacumicin B, show activity against a variety of bacterial pathogens and in particular against C. difficile (Antimicrob. Agents Chemother. 1991, 1108-1111). Because Tiacumicin B shows promising activity against C. difficile, it is useful in the treatment of bacterial infections, especially those of the gastrointestinal tract, in humans. Examples of such treatments include but are not limited to treatment of colitis and treatment of irritable bowel syndrome. Tiacumicin antibiotics are described in U.S. Pat. No. 4,918,174, J. Antibiotics 1987, 40: 575-588, J. Antibiotics 1987, 40: 567-574, J. Liquid Chromatography 1988, 11: 191-201, Antimicrobial Agents and Chemotherapy 1991, 35: 1108-1111, U.S. Pat. No. 5,583,115, U.S. Pat. No. 5,767,096, U.S. Pat. No. 7,906,489, U.S. Pat. No. 7,863,249 and U.S. Pat. No. 8,044,030. Related compounds are the Lipiarmycin antibiotics (J. Chem. Soc. Perkin Trans. I, 1987, 1353-1359 and J. Antibiotics 1988, 41: 308-315) and the Clostomicin antibiotics (J. Antibiotics 1986, 39: 1407-1412).

### SUMMARY OF THE INVENTION

The invention encompasses a method of preventing a Clostridium infection in a human subject who is at high risk of developing a Clostridium infection, comprising orally administering to the human subject an amount of a compound of Formula I:

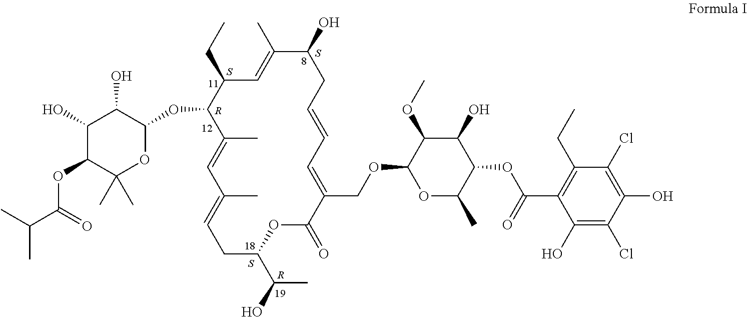

effective to inhibit the proliferation of Clostridium in the gastrointestinal tract of the human subject.

In some embodiments, the subject is a hematopoietic stem cell transplant (HSCT) patient and may also be suffering from Acute Lymphoblastic Leukemia (ALL) or Acute Myeloid Leukemia (AML). In some embodiments, the subject is receiving treatment in an intensive care unit (ICU). In some embodiments, the subject has only received radiation therapy, while in other embodiments, the subject has only received chemotherapy.

In some embodiments, the Clostridium infection is caused by C. difficile. In some embodiments, the subject is further receiving concomitant antibiotic therapy to prevent and/or treat an infection other than Clostridium. In some embodiments, the subject previously was treated for a Clostridium infection. In some embodiments, the compound of formula I prevents the Clostridium infection.

In some embodiments of the method of the invention, the compound of Formula I is administered as a pharmaceutical composition. In some embodiments, the pharmaceutical composition further comprises butylated hydroxy toluene. In some embodiments, the pharmaceutical composition is administered orally. In some embodiments, the pharmaceutical composition contains at least 93% of the compound of Formula I.

In some embodiments, the compound of Formula I is present as a crystalline polymorph with an X-ray diffraction pattern with peaks at diffraction angles 2θ 7.7°, 15.0° and 18.8°±0.2° as set forth in FIG. 1. In some embodiments, the X-ray diffraction pattern is as set forth in FIG. 1.

### BRIEF DESCRIPTION OF FIGURE

FIG. 1 shows XRD data for a crystalline form of the compound of Formula I.

### DETAILED DESCRIPTION OF THE INVENTION

The present invention relates to a method of preventing a Clostridium difficile infection in a human subject who is at high risk of developing this disease, such as an HSCT patient, comprising administering to the subject an amount of a compound of Formula I below effective to inhibit proliferation of Clostridium difficile in the human subject:

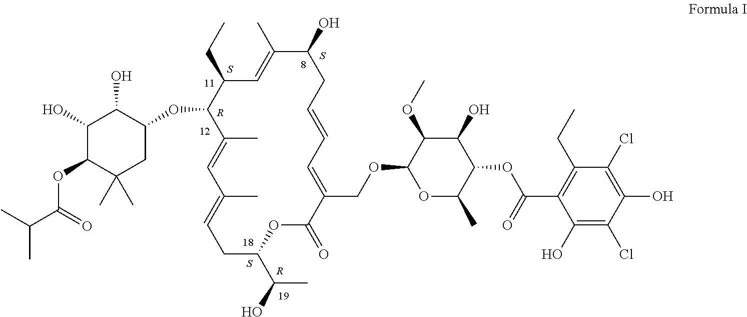

The present also invention relates to a method of treating a Clostridium difficile infection in a human subject who is at high risk of developing this disease, such as an HSCT patient, comprising administering to the subject an amount of a compound of Formula I effective to inhibit proliferation of Clostridium difficile in the human subject.

Human subjects which are subjected to the methods of the invention are considered high risk patients of developing CDI because of their underlying disease, prolonged hospitalization, especially in an Intensive Care Unit (ICU), and/or therapy with broad-spectrum antibiotics, which makes them more susceptible to opportunistic infections (including CDI) which usually do not occur in healthy human subjects. Prolonged stays in hospital including an ICU which renders the subject more susceptible to CDI, therefore the methods of the invention include administration of the compound of formula I to hospital patients, including ICU patients, who have been in an ICU for at least one week, two weeks, three weeks or even up to, and exceeding, one month.

In some embodiments the high risk patients are defined as patients who are predisposed to gastrointestinal infection with C. difficile because they are being treated with broad spectrum antibiotics when undergoing various medical procedures to treat their underlying diseases caused by one or more bacterial infections. In some embodiments the high risk patients are defined as patients who are given broad spectrum antibiotics as prophylaxis when undergoing a surgical procedure, or are on mechanical ventilation in an intensive care unit.

In other embodiments, these high risk patients are being treated for a selected disease such as cancer which requires chemotherapy and/or radiation therapy, such therapies resulting in susceptibility to bacterial infections. In certain embodiments, these high risk patients also have undergone surgery to excise a tumor, the surgical procedure further predisposing them to opportunistic infections commonly associated with surgery. In certain embodiments the cancer is selected from the group consisting of Leukemia including Acute Lymphoblastic Leukemia (ALL), Acute Myeloid Leukemia (AML), Chronic Lymphocytic Leukemia (CLL), Chronic Myelogenous Leukemia (CML); Lymphoma including AIDS-Related Cancers such as Kaposi Sarcoma and Lymphoma, Burkitt Lymphoma, Cutaneous T-Cell Lymphoma, Hodgkin Lymphoma, Non-Hodgkin Lymphoma, and Chronic Myeloproliferative Disorders. In some embodiments, the cancer is selected from a cancer of the gastrointestinal tract including Gastric (Stomach) Cancer, Colon Carcinoma, Liver Cancer, including Hepatocellular Carcinoma and Hepatoma, Gallbladder Cancer, Pancreatic Cancer and Gastrointestinal stromal tumors.

CDI is an ongoing challenge to patients in these settings. Thus, the administration of the compound of Formula I to these patients to prevent the onset of CDI is encompassed in the present invention. Exemplary embodiments of the methods of the invention therefore include prevention and/or treatment of CDI in a patient in an intensive care unit by administering an effective amount of the compound of formula I. In some embodiments these patients are being treated for cancer with chemotherapy and/or radiation therapy. In some embodiments the cancer is malignant cancer.

As used herein, “fidaxomicin” refers to the therapeutically active agent tested in the Examples described herein that comprises the compound of Formula I. In some embodiments, fidaxomicin may also include other tiacumicin related compounds as described herein. In further embodiments, fidaxomicin may include crystalline polymorphic and/or amorphous forms of the compound of Formula I as described in the Examples herein.

As used herein, the term “treatment” indicates a procedure which is designed ameliorate one or more causes, symptoms, or untoward effects of a bacterial infection in a subject. Likewise, the term “treat” or “treating” is used to indicate performing a treatment. The treatment can, but need not, cure the subject, i.e., remove the cause(s), or remove entirely the symptom(s) and/or untoward effect(s) of the bacterial infection in the subject. Thus, a treatment may include treating a human subject to inhibit the growth or proliferation of Clostridium difficile, in the subject, or it may attenuate symptoms such as, but not limited to, diarrhea, fever, cramps, dehydration and peritonitis, or may include removing or decreasing the severity of the root cause of the Clostridium difficile infection in the human subject. Treatment of a bacterial infection also includes treating after-arising symptoms that are related to the initial infection, such as diarrhea, fever, cramps, dehydration and peritonitis.

As used herein, the term “prevention” indicates a procedure which is designed inhibit the onset of one or more causes, symptoms, or untoward effects of a bacterial infection in a subject. Likewise, the term “prevent” or “preventing” is also used to indicate a procedure which is designed to inhibit the onset of one or more causes, symptoms, or untoward effects of a bacterial infection in a subject. The prevention can block the onset of the cause (e.g. proliferation of bacteria, including but not limited to, C. difficile), or block entirely any symptoms and/or untoward effects of the bacterial infection in the subject. Thus, prevention may include administration of a compound of formula I to a human subject to inhibit the growth or proliferation of Clostridium difficile, in the gastrointestinal tract of the subject, or it may block the onset of symptoms such as, but not limited to, diarrhea, fever, cramps, dehydration and peritonitis.

As used herein, the term “subject” is used interchangeably with the term “patient” and is used to mean a human. As used herein, the term “high risk subject” is used to mean a human who is at risk of developing CDI. In some embodiments, the subjects are HSCT patients, cancer patients, including those undergoing chemotherapy and/or radiation therapy. In some embodiments, the subjects are patients in an intensive care unit given their advanced disease state. In some embodiments, the patient in the intensive care unit is on life support such as mechanical ventilation, further increasing the risk for infection.

A “bacterial infection” or “infection” are used interchangeably and is used herein as it is described in the art, and the phrase is also used herein to include conditions or symptoms associated with the bacterial infection. In one embodiment, the bacterial infection is an infection of Clostridium difficile (C. difficile), Staphylococcus species, including but not limited to methicillin-resistant S. aureus (MRSA), Enterococcus species including but not limited to vancomycin-resistant Enterococci (VRE) or Clostridium perfringens (C. perfringens). The bacterial infection can be in any system, organ, tissue or area of the subject, such as but not limited to, gastrointestinal tract including upper and/or lower portions thereof. In some embodiments, the infection is a C. difficile infection of the GI tract.

In one embodiment, the infection is a first-time gastrointestinal (GI) infection of C. difficile, while in another embodiment, the infection is a recurring (GI) infection of C. difficile. As used herein, a recurring infection is an infection wherein the infection or the symptoms thereof occurs at an additional point in time, including more than once. The previous or initial infection or symptoms thereof may or may not have been treated prior to the reoccurrence of the infection or symptoms thereof. In one embodiment, the human subject was not previously treated for the recurrent GI infection of C. difficile. In another embodiment, the subject was previously treated for the recurrent GI infection of C. difficile.

In some embodiments, the subject was not previously treated for the GI infection of C. difficile, while in another embodiment, the subject was previously treated. In these cases, the subject was treated with a composition or substance not including the compound of Formula I. Substances or compositions that may be used in these embodiments include any known antibiotic, including but not limited to, metronidazole, vancomycin, fusidic acid, rifaximin, bacitracin, tetracyclines, fluoroquinolones and/or teicoplanin. In other embodiments, the subject was previously treated for the GI infection of C. difficile and was treated with a composition or substance comprising compound of Formula I.

Other bacterial infections and disorders related to such infections include but are not limited to disorders associated with the use of antibiotics, chemotherapy, or radiation therapy, including, but not limited to, colitis, for example, pseudo-membranous colitis or antibiotic associated diarrhea. More specifically, antibiotic-associated diarrhea caused by toxin producing strains of C. difficile, S. aureus including methicillin-resistant S. aureus, and C. perfringens.

Methods of treating or preventing a bacterial infection or a recurring infection described herein comprise administering a pharmaceutically effective amount of the compound of Formula I to a high risk patient. As used herein, the term “administer” and “administering” are used to mean introducing the compound of Formula I to a subject. When administration is for the purpose of treatment, the substance is provided at, or after the onset of, a symptom of a bacterial infection. The therapeutic administration of this substance serves to attenuate any symptom, or prevent additional symptoms from arising. When administration is for the purposes of preventing or reducing the likelihood a bacterial infection or a recurrent (“prophylactic administration”), the substance is provided in advance of any visible or detectable symptom, such as after the symptoms of the initial infection. The prophylactic administration of the substance serves to attenuate subsequently arising symptoms or prevent or reduce the likelihood of the symptoms from arising altogether. Accordingly, the compound of Formula I may be used for the prevention of one disease or disorder and concurrently treating another.

The route of administration of the compound includes, but is not limited to, oral (such as a tablet, capsule or oral suspension), topical or rectal (e.g. enema).

Furthermore, the methods of treating or preventing a bacterial infection of the present invention also relate to co-administering one or more substances in addition to the compound of Formula I to the subject. The term “co-administer” indicates that each of at least two compounds are administered during a time frame wherein the respective periods of biological activity or effects overlap. Thus, the term includes sequential as well as coextensive administration of compounds. And similar to administering compounds, co-administration of more than one substance can be for therapeutic and/or prophylactic purposes. If more than one substance or compound is co-administered, the routes of administration of the two or more substances need not be the same. The scope of the invention is not limited by the identity of the substance which may be co-administered with the compound of Formula I. For example, the compound of Formula I may be co-administered with other pharmaceutically active substances, such as any known chemotherapeutic. Alternatively, compositions comprising the compound of Formula I may be co-administered with fluids or other substances that are capable of alleviating, attenuating, preventing or removing symptoms in a subject suffering from, exhibiting the symptoms of, or at risk of suffering from a bacterial infection. Types of fluid that can be co-administered with the compound of Formula I should be specific to the circumstances surrounding the particular subject that is suffering from, exhibiting the symptoms of, or at risk of suffering from a bacterial infection. For example, fluids that may be co-administered with the compound of Formula I include but are not limited to, electrolytes and/or water, salt solutions, such as sodium chloride and sodium bicarbonate, as well as whole blood, plasma, serum, serum albumin and colloid solutions.

As used herein and unless otherwise indicated, the phrase “therapeutically effective amount” (or “pharmaceutically effective amount”) of the compound of Formula I or a pharmaceutically acceptable salt or prodrug thereof is measured by the therapeutic effectiveness of a compound of the invention, wherein at least one adverse effect of a disorder is ameliorated or alleviated. In one embodiment, the term “therapeutically effective amount” means an amount of the compound of Formula I that is sufficient to provide the desired local or systemic effect and performance at a reasonable benefit/risk ratio attending any medical treatment. The response to the therapeutically effective amount may be a cellular, organ or tissue-specific response, or system (e.g., GI tract) systemic response. In one embodiment, the phrase “therapeutically effective amount” of a composition of the invention is measured by the therapeutic effectiveness of a compound of the invention to alleviate at least one symptom associated with bacterial (e.g., C. difficile) infections. Examples of therapeutically effective amounts include, but are not limited to those in the Examples section herein.

As used herein and unless otherwise indicated, the term “binders” refers to agents used to impart cohesive qualities to the powdered material. Binders, or “granulators” as they are sometimes known, impart cohesiveness to the tablet formulation, which insures the tablet remaining intact after compression, as well as improving the free-flowing qualities by the formulation of granules of desired hardness and size. Materials commonly used as binders include starch, gelatin, sugars, such as sucrose, glucose, dextrose, molasses, and lactose, natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, larch arabogalactan and the like.

As used herein and unless otherwise indicated, the term “compounds of the invention” means, collectively, a compound of Formula I and/or pharmaceutically acceptable salts, solvates, hydrates, amorphous forms and crystalline polymorphs thereof. The compounds of the invention are identified herein by their chemical structure and/or chemical name. Where a compound is referred to by both a chemical structure and a chemical name, and that chemical structure and chemical name conflict, the chemical structure is determinative of the compound's identity. The compounds of the invention may contain one or more chiral centers and/or double bonds and may therefore exist as stereoisomers, such as double-bond isomers (i.e. geometric isomers), enantiomers, or diastereomers. According to the invention, the chemical structures depicted herein, and therefore the compounds of the invention, encompass all of the corresponding compound's enantiomers and stereoisomers, that is, both the stereomerically pure form (e.g. geometrically pure, enantiomerically pure, or diastereomerically pure) and enantiomeric and stereoisomeric mixtures, and solvates and/or hydrates thereof. Enantiomeric and stereoisomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well known asymmetric synthetic methods.

In some embodiments, the pharmaceutical compositions used in the methods of the present invention comprise the compound of Formula I that is substantially stereomerically pure. In specific embodiments, the pharmaceutical compositions comprise the compound of Formula I that is at least about 75% pure, 80%, 85%, 90%, 91%, 92%, 93%, 94%, 95%, 96%, 97%, 98% or 99% pure, i.e., free from other stereoisomers, diastereoisomers, enantiomers, etc.

As used herein, and unless otherwise indicated, the terms “stereomerically pure” and “substantially stereomerically pure” are used interchangeably and mean one stereoisomer of a compound or a composition that comprises one stereoisomer of a compound and is substantially free of other stereoisomer(s) of that compound. For example, a stereomerically pure compound or composition of a compound having one chiral center will be substantially free of the opposite enantiomer of the compound. A stereomerically pure compound or composition of a compound having two chiral centers will be substantially free of other diastereomers of the compound. A typical stereomerically pure compound comprises at least about 80% by weight of one stereoisomer of the compound, i.e., free from other stereoisomers, diastereoisomers, enantiomers, etc. and about 20% or less by weight of other stereoisomers of the compound, more specifically at least about 90% by weight of one stereoisomer of the compound and about 10% or less by weight of the other stereoisomers of the compound, even more specifically, at least about 95% by weight of one stereoisomer of the compound and about 5% or less by weight of the other stereoisomers of the compound, and more specifically, at least about 97% by weight of one stereoisomer of the compound and about 3% or less by weight of the other stereoisomers of the compound.

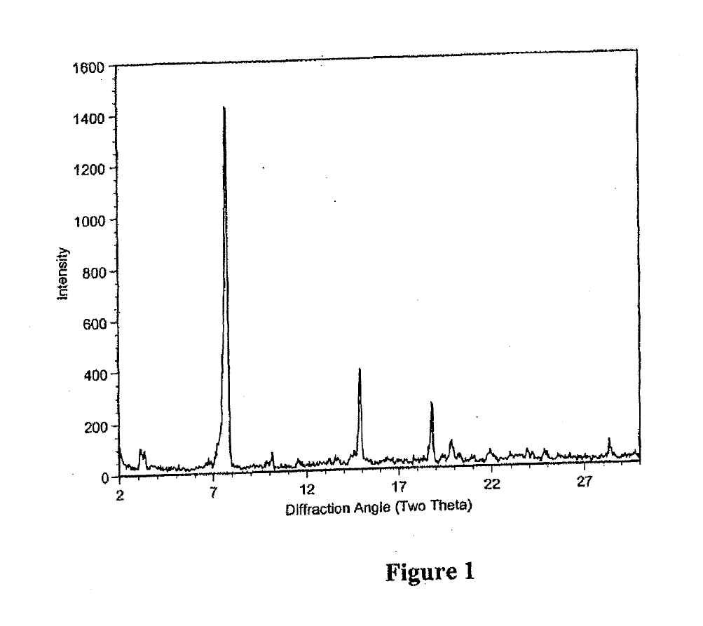

As used herein and unless otherwise indicated, the term “crystalline polymorph” refers to the compound of Formula I that exists in several distinct forms (e.g., crystalline, amorphous), the invention encompasses all of these forms. In another embodiment, a pharmaceutically acceptable polymorph of a Compound of Formula I exhibits a representative powder diffraction pattern comprising at least peaks at the following diffraction angles 2Θ of 7.7°, 15.0°, and 18.8°±0.04, or ±0.1, or ±0.15, or ±0.2, as shown in FIG. 1. In another embodiment, a pharmaceutically acceptable polymorph of a Compound of Formula I exhibits a representative powder diffraction pattern comprising at least peaks at the following diffraction angles 2Θ of 7.6°, 15.4°, and 18.8°±0.04, or ±0.1, or ±0.15, or ±0.2, as shown in FIG. 2.

Methods of preparing and characterizing select embodiments of pharmaceutically acceptable polymorphs are found in U.S. Pat. No. 7,863,249.

As used herein and unless otherwise indicated, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like.

As used herein and unless otherwise indicated, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

The term “MIC” or “minimum inhibitory concentration” refers to the lowest concentration of an antibiotic that is needed to inhibit growth of a bacterial isolate in vitro. A common method for determining the MIC of an antibiotic is to prepare several tubes containing serial dilutions of the antibiotic, that are then inoculated with the bacterial isolate of interest. The MIC of an antibiotic can be determined from the tube with the lowest concentration that shows no turbidity (no growth). The term “MIC50” refers to the lowest concentration of antibiotic required to inhibit the growth of 50% of the bacterial strains tested within a given bacterial species. The term “MIC90” refers to the lowest concentration of antibiotic required to inhibit the growth of 90% of the bacterial strains tested within a given bacterial species.

As used herein and unless otherwise indicated, “pharmaceutically acceptable” refers to materials and compositions that are physiologically tolerable and do not typically produce an allergic or similar untoward reaction, such as gastric upset, dizziness and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein and unless otherwise indicated, the term “hydrate” means the compound of Formula I that further includes a stoichiometric or non-stoichiometric amount of water bound by non-covalent intermolecular forces.

The phrase “pharmaceutically acceptable salt(s)” as used herein includes but is not limited to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds, included in the present compositions, which are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

In some embodiments, the methods of the invention encompass administering pharmaceutical compositions comprising a first polymorph of the compound of Formula I, a second polymorph of the compound of Formula I, other polymorphic forms, amorphous form or mixtures thereof in varying amounts. Certain embodiments of the methods of the present invention may also comprise administering pharmaceutical compositions that containing these mixtures for treating CDI in HSCT patients.

The present compositions, which comprise one or more crystalline polymorph or amorphous form of the compound of Formula I may be administered by any convenient route for delivery to the GI tract, for example, peroral administration, by infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a composition of the invention. In certain embodiments, more than one compound of Formula I is administered to a patient. Methods of administration include but are not limited to oral, rectally, or topically, particularly to the GI tract. The mode of administration is left to the discretion of the practitioner, and will depend in part upon the site of the medical condition. In most instances, administration will result in the release of the crystalline polymorph or amorphous form of the compound of Formula I into the GI tract.

In specific embodiments, it may be desirable to administer one or more crystalline polymorph or amorphous form of the compound of Formula I locally to the area in need of treatment. This may be achieved, for example, and not by way of limitation, by local infusion during surgery, topical application, by injection, by means of a catheter, by means of a suppository, or by means of an implant, said implant being of a porous, non-porous, or gelatinous material, including membranes, such as sialastic membranes, or fibers.

The present compositions will contain a therapeutically effective amount of a crystalline polymorph or amorphous form of the compound of Formula I, optionally more than one crystalline polymorph or amorphous form of the compound of Formula I, for example in purified form, together with a suitable amount of a pharmaceutically acceptable vehicle so as to provide the form for proper administration to the patient.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for oral or topical use. In one embodiment, the pharmaceutically acceptable vehicle is a capsule. Other examples of suitable pharmaceutical vehicles are described in Remington's The Science and Practice of Pharmacy, Berringer (Ed) (2006). The pharmaceutical compositions may contain preserving agents, solubilising agents, stabilising agents, wetting agents, emulsifiers, sweeteners, colorants, odorants, salts, buffers, coating agents or antioxidants, such as, but not limited to butylated hydroxytoluene (BHT). They may also contain therapeutically active agents in addition to the substance of the present invention.

In one embodiment, the compositions of the invention are administered orally. Compositions for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered crystalline polymorph or amorphous form of the compound of Formula I. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a crystalline polymorph or amorphous form of the compound of Formula I that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. Suitable dosage ranges for oral administration, however, are generally from about 0.001 milligram to 1000 milligrams of the compound of Formula I per kilogram body weight. In one embodiment, the oral dose is about 0.01 milligram to about 500 milligrams per kilogram body weight, or from about 0.1 milligram to about 100 milligrams per kilogram body weight, or from about 0.5 milligram to about 50 milligrams per kilogram body weight. In a specific embodiment, the oral dose is from about 1 milligram to about 10 milligrams per kilogram body weight. In a more specific embodiment, the oral dose is about 1 milligram of a crystalline polymorph or amorphous form of the compound of Formula I per kilogram body weight. The dosage amounts described herein refer to total amounts administered; that is, if more than one compound is administered, the preferred dosages correspond to the total amount of the compounds of the invention administered. The oral compositions described herein may contain from about 10% to about 95% active ingredient by weight, and the oral compositions may be dosed 1, 2, 3, 4, 5 or more times daily. Preferably, the oral compositions described herein are administered to a patient in a dosage range from about 50 mg to about 400 mg per day for 10 to 50 days. More preferably, the oral compositions described herein are administered to a patient once a day at a dose of about 200 mg for 40 days.

Suitable dosage ranges for intranasal administration are generally from about 0.01 pg/kg body weight to about 1 mg/kg body weight of the compound of Formula I. Suppositories generally contain from about 0.01 milligram to about 50 milligrams of the compound of Formula I per kilogram body weight and comprise active ingredient in the range of from about 0.5% to about 10% by weight. Recommended dosages for intradermal, intramuscular, intraperitoneal, epidural, sublingual, intracerebral, intravaginal, transdermal administration or administration by inhalation are in the range of from about 0.001 milligram to about 1000 milligrams per kilogram of body weight of the compound of Formula I. Suitable doses of the compounds of the invention for topical administration are in the range of from about 0.001 milligram to about 1 milligram of the compound of Formula I, depending on the area to which the compound is administered. Effective doses may be extrapolated from dose-response curves derived from in vitro or animal model test systems. Such animal models and systems are well known in the art.

Methods of manufacturing the compound of Formula I, including select polymorphs thereof are disclosed in U.S. Pat. No. 7,378,508.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1

### Production of Compound of Formula I

The compound of Formula I can be produced by fermentation. Cultivation with a mutant form derived from Dactylosporangium aurantiacum subspecies hamdenensis AB 718C-41 NRRL 18085 for the production was carried out in a medium containing carbon sources, inorganic salts and other organic ingredients with one or more absorbents under proper aeration conditions and mixing in a sterile environment. The production method is disclosed in U.S. Pat. No. 7,507,564.

The nutrient medium comprises from about 0.5 to about 15% of the adsorbent by weight. In one embodiment, the absorbent is an adsorbent substance, such as a resin. Examples of absorbent substances include but are not limited to SP285, Amberlite®, XAD 16, XAD 16HP, XAD2, XAD7HP, XADI 180, XAD 1600, IRC50, or Duolite® XAD761. The nutrient medium can comprise the following combination based on weight: from about 0.2% to about 10% of glucose, from about 0.02% to about 0.5% of K₂HPO₄, from about 0.02% to about 0.5% of MgSO₄.7H₂O, from about 0.01% to about 0.3% of KCl, from about 0.1% to about 2% of CaCO₃ and from about 0.5% to about 15% of SP285 resin. The culturing step was conducted at a temperature from about 25° C. to about 35° C. and at a pH from about 6.0 to about 8.0.

Upon completion of fermentation, the solid mass (including the adsorbent resin) was separated from the broth. The products were extracted with organic solvents such as, for example, ethyl acetate then concentrated under reduced pressure.

### Example 2

### Purification of Compound of Formula I

After the fermentation in Example 1, the crude material was purified by HPLC. The collected fractions containing about 90-99% of compound of Formula I were combined. The solid was crystallized to the desired crystalline form to produce the pharmaceutical composition (fidaxomicin) using methanol and water solvent system as set forth in U.S. Pat. No. 7,863,249. HPLC analysis showed fidaxomicin to contain about >93% of compound of Formula I as a major component and a mixture of tiacumicins as the minor component.

### Example 3

### Clinical Study

A multi-center study utilizes a randomized, double-blind design to compare the safety and efficacy of fidaxomicin as Clostridium difficile-associated diarrhea or CDI prophylaxis in individuals undergoing hematopoietic stem cell transplant or HSCT. Subjects are randomized to one of two treatment groups in a 1:1 ratio (1 active:1 placebo) and receive fidaxomicin 200 mg or placebo administered orally once daily from the initiation of a standard of care medication (fluoroquinolone prophylaxis) prior to transplantation until 7 days after completion of such a treatment administration.

Blinded study drug treatment is initiated with prophylactic fluoroquinolones according to standard of care practices. During the study, patients are managed and followed per institutional guidelines for the management of HSCT patients as documented in the institutional guidelines. Patients receive appropriate treatment for their underlying diseases, as well as any complications resulting from the transplantation, based on institutional protocols and the judgment of the treating physician. With the exception of unapproved investigational agents, any clinically indicated concomitant medication will be allowed.

Subjects are monitored daily during the blinded treatment period for number of bowel movements, their consistency (solid, mushy, liquid) and stool toxin testing as indicated. After discontinuation of blinded study drug, subjects are contacted twice weekly through 30 days post treatment to evaluate signs and symptoms of CDI and then weekly through 60 days post-treatment of study drug prophylaxis for evidence of CDI. If a subject reports signs and symptoms of suspected CDI, they are instructed to return to the clinical site for C. difficile testing (C. difficile toxin assay by PCR testing).

Although the present invention has been described in detail with reference to examples above, it is understood that various modifications can be made without departing from the spirit of the invention. Accordingly, the invention is limited only by the following claims. All patents, published patent applications, and other published references cited herein are incorporated by reference in their entirety.

1. A method of preventing a Clostridium infection in a human subject receiving treatment in an intensive care setting and who is at high risk of developing a Clostridium infection, comprising orally administering to the human subject an amount of a compound of Formula I: 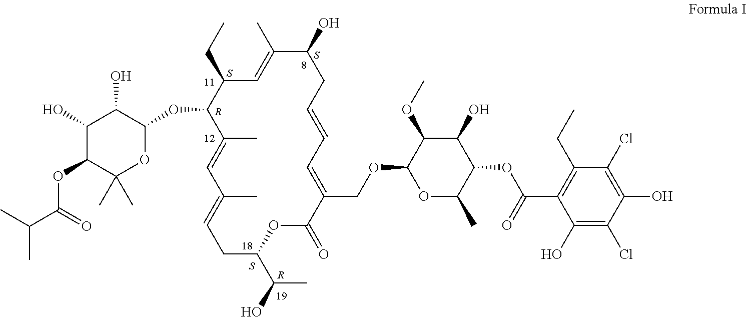
effective to inhibit the proliferation of Clostridium in the gastrointestinal tract of the human subject.

2. The method of claim 1 wherein the subject is a hematopoietic stem cell transplant (HSCT) patient.

3. (canceled)

4. The method of claim 2 wherein the subject is also suffering from Acute Lymphoblastic Leukemia (ALL) or Acute Myeloid Leukemia (AML).

5. The method of claim 1 wherein the Clostridium infection is caused by C. difficile.

6. The method of claim 1 wherein the subject is further receiving concomitant antibiotic therapy to prevent and/or treat an infection other than Clostridium.

7. The method of claim 1 wherein the subject previously was treated for a Clostridium infection.

8. The method of claim 1 wherein the human subject has only received radiation therapy.

9. The method of claim 1 wherein the human subject has only received chemotherapy.

10. The method of claim 1 wherein the compound of Formula I is administered as a pharmaceutical composition.

11. The method of claim 10 wherein the pharmaceutical composition further comprises butylated hydroxy toluene.

12. The method of claim 10 wherein the pharmaceutical composition is administered orally.

13. The method of claim 12 wherein the pharmaceutical composition is to a patient in a dosage range from about 50 mg to about 400 mg per day for 10 to 50 days.

14. The method of claim 13 wherein the pharmaceutical composition is administered to a patient once a day at a dose of about 200 mg for 40 days.

15. The method of claim 10 wherein the pharmaceutical composition contains at least 93% of the compound of Formula I.

16. The method of claim 1 wherein the compound of Formula I is present as a crystalline polymorph with an X-ray diffraction pattern with peaks at diffraction angles 2θ 7.7°, 15.0° and 18.8°±0.2° as set forth in FIG. 1.

17. The method of claim 16 wherein the X-ray diffraction pattern is as set forth in FIG. 1.
